(19) United States
(12) Patent Application Publication
(10) Pub. No.: US 2003/0072780 A1
(43) Pub. Date: Apr. 17, 2003

(54) Fluid-to-powder compositions

(76) Inventors: Mirela Cristina Ionita-Manzatu, Old Bethpage, NY (US); Jennifer Oko, Stony Brook, NY (US); John D. Dreher, Sayville, NY (US); Rachel J. Painter, Brooklyn, NY (US); Isaac D. Cohen, Brooklyn, NY (US)

Correspondence Address: Karen A. Lowney, Eaq., ESTEE LAUDER COMPANIES, 125 Pinelawn Road, Melville, NY, 11747, US

(21) Appl. No.: 10/201,364

(22) Filed: Jul. 23, 2002

Related U.S. Application Data
(60) Provisional application No. 60/317,906, filed on Sep. 6, 2001.

(51) Int. Cl. A61K 9/14; A61K 7/00

(52) U.S. Cl. 424/401

(57) ABSTRACT

The invention relates to fluid-to-powder cosmetic or pharmaceutical compositions for topical application to skin, the composition comprising at least one polyflurorinated solvent gelled with a fumed silica.

### FIELD OF THE INVENTION

The present invention relates to cosmetic and pharmaceutical compositions. More specifically, the invention a novel vehicle for cosmetic and pharmaceutical compositions.

### BACKGROUND OF THE INVENTION

Compositions for topical application to the skin come in a variety of different forms. The range of product forms may run from dry powders to water thin liquids, to thicker creams and lotions, to semi-solid to solid sticks. Each product form has its own certain advantages, and the choice of product form may be governed by the nature of the ultimate purpose of the product and/or the chemical identity of the crucial ingredients of the composition as a whole. Consumer perception also plays a significant role in the choice of product form: for example, consumers often perceive a product that is clear as being cooling or refreshing, and therefore, a clear gel or stick may be preferentially chosen for a product which wishes to project such an image.

Creams and lotions are usually perceived by a consumer as being rich and elegant; in their best embodiments, they go on smoothly, are easily distributed on the skin, remaining where they are placed, and rub in invisibly, leaving a moisturized, pampered feel on the skin. However, the emollient materials that permit this ease of application and luxurious feel may in some users leave the feeling of a greasy residue on the skin, and in warm weather may seem too heavy for regular use, thereby depriving the user of the potential benefits under all conditions. Other types of vehicles, such as powders, can avoid some of these issues, because they go on dry, and have a light, cool feel on the skin. However, powders also have their drawbacks, particularly in the ease of application: because of the looseness of the powder structure, application is hard to control, and without due care, a significant portion of the powder will end up on the floor as well as on the skin of the user. It would, therefore, be a benefit to users to have available a product that combines all the elegant feel and application of the cream/lotion type of product with the coolness of a powder product.

In recognition of this desirable combination, a number of cosmetic products that go on in fluid form, and transform to powder on the skin, are currently available. Typically, such products, in order to give a quick transformation from liquid to powder, are based on a volatile carrier, usually an alcohol or volatile hydrocarbon, which evaporates quickly from the skin. However, such products themselves have disadvantages, in that these volatiles can strip the skin, and also leave an unpleasant, whitish residue on the skin upon drying. In addition, such materials are often highly flammable, and therefore, present difficulties in the manufacturing process as well. The gellant for such formulas is also ordinarily a starch, such as tapioca or potato starch, which can leave a gritty feel on the skin. Overall, because of the selected components, such products also do not ordinarily have as creamy a feel as might be desired, nor is their level of evaporation upon application so thorough as to leave a substantially completely dry product. There thus remains a need for a liquid-to-powder product that provides all the desired characteristics of a luxurious fluid product and cooling powder product, without the disadvantages that have typically been associated with such products. The present invention now fulfills such a need.

### SUMMARY OF THE INVENTION

The invention relates to topical compositions for application to the skin, the composition being a cream or lotion, comprising at least one polyfluorinated solvent gelled by a fumed silica. The compositions of the invention apply to the skin as a fluid, e.g., as a cream or lotion, and upon rubbing on the skin, convert virtually instantaneously to a dry powder. The compositions of the invention provide a suitable vehicle for makeup and skin care products, as well as for pharmaceutical actives.

### DETAILED DESCRIPTION OF THE INVENTION

It has been unexpectedly discovered that by gelling a polyfluorinated solvent with a fumed silica, it is possible to produce a product that initially has the elegant, smooth spreading properties of a cream or lotion, but which upon application to the skin, converts to a dry, silky-feeling powder. The polyfluorinated solvents of the invention, like the traditional solvents used for this purpose, evaporate fairly quickly, leaving a powder residue on the skin. However, unlike many of the other types of solvents, the polyfluorinated solvents of the invention are relatively inert, thereby avoiding potential problems in manufacturing, and further do not strip the skin. The solvents of the invention have previously been recognized for their quick-drying properties, but to the best of Applicants' knowledge, they have not been previously used in a liquid to powder composition.

The solvent of the invention can be selected from among several different polyfluorinated solvents of this type. In a preferred embodiment, the solvent may be a polyfluorocycloalkane. Compounds of this type are commercially available from F2 Chemicals, Ltd. under the trade name FLUTEC™. These products come in a variety of forms, which differ from each other in molecular weight and viscosity, and relative volatility. Generally speaking, the molecular weights range from about 300 to about 800, with vapor pressures in the range of from <0.1 mbar up to about 500 mbar, and boiling points in the range of from about 45° C. to about 260° C. The preferred compounds of this type are those in the mid-range of viscosity and volatility, i.e. those having a molecular weight of between about 400 to about 650, vapor pressures of about <1 to about 50 mbar, and boiling points ranging from about 100° C. to about 220° C. Particularly preferred compounds of this type are perfluoro-1,3-dimethylcyclohexane, known as FLUTEC™ PC3, perfluorodecalin, sold as FLUTEC™ PC6, perfluoromethyldecalin, sold as FLUTEC™ PP9, and perfluorohydrophenanthrene, sold as FLUTEC™ PC11.

Although the perfluorocycloalkanes are particularly preferred, there are other groups of polyfluorinated solvent that can also be used. Another group of very useful fluorinated solvents are hydrofluoroethers. Compounds of this type are disclosed, for example, in FR 2771290, the contents of which are incorporated herein by reference. The formula of such hydrofluoroethers is as follows:

CH₃—(CH₂)ₙ—[Z]ₜ—X—CF₃

wherein t is 0 or 1; n is 0, 1, 2 or 3; X is a linear or branched divalent perfluoroalkane radical having 2 to 5 carbon atoms, and Z is O, S or NR, wherein R is hydrogen, or a radical —(CH₂)ₙ—CH₃ or —(CF₂)ₘ—CF₃, where m is 2, 3, 4 or 5. Preferably, Z is O, and t is 1. Specific examples of these types of compounds are methoxynonafluorobutane, ethoxynonafluorobutane, or propoxy-undecafluoropentane. Such compounds are available commercially from 3M or Archimex under the designation “HFE”.

Also included in the polyfluorinated solvents are perfluoromorpholine compounds having the formula:

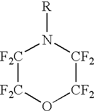

wherein R is a C₁-C₄ perfluoroalkane radical. Examples of such compounds are 4-trifluoromethylperfluoromorpholine and 4-pentafluoroethylperfluoromorpholine.

An additional example of polyfluorinated solvents are perfluoroalkanes having the formula

CF₃—(CF₂)ₙ—CF₃

wherein n is an integer from 2-6. Examples of such compounds include dodecafluoropentane and tetradecafluorohexane.

Preferably, the polyfluorinated solvents are used alone as the fluid base, or they may be combined with one or more other volatile solvents. Examples of other solvents include, but are not limited to, both cyclic and linear silicones, such as octamethylcyclotetrasiloxane and decamethylcyclopentasiloxane; or straight or branched chain hydrocarbons having from 8-20 carbon atoms, such as decane, dodecane, tridecane, tetradecane, and C8-20 isoparaffins. If a second solvent is used, a preferred companion solvent is a relatively volatile, low viscosity dimethicone. An example of a particularly useful dimethicones are available from Dow Corning under the trade name DC 200, with a range of viscosities between 0.65 and 100 cs, preferably between 5 and 20 cs, which may be used singly, or in combination. In formulating the fluid base, the solvent portion overall, whether a polyfluorinated solvent alone, or in combination, call comprise from about 40 to about 98% by weight of the composition. preferably about 50% to about 90%, more preferably about 65% to about 90%. In a preferred embodiment, the polyfluorinated solvent is used alone in the aforementioned amounts. When the polyfluorinated solvent is combined with a secondary volatile solvent, they may be combined in any proportion. However, it is preferred., particularly when dimethicone is the secondary solvent, to use larger quantities of the secondary solvent. As an example, the polyfluorinated solvent will preferably be used in an amount of about 5 to about 40%, by weight of the composition, and dimethicone used in an amount of from about 50 to about 85% by weight.

To form the compositions of the invention, the polyfluorinated solvents are combined with a fumed silica(also called silica silylate) as a gellant. By “fumed silica” it is meant those high-surface area powdered silicas prepared by a pyrogenic process, e.g., during burning silicon tetrachloride in air (i.e., by the flame hydrolysis of silicon tetrachloride) and has a purity of 99.8% or greater. In this process, submicron sized molten spheres of silica collide and fuse to form three dimensional, branched, chain-like aggregates, of approximately 0.1 to 0.5 microns in length. Cooling takes place very quickly, limiting the particle growth and ensuring the fumed silica is amorphous. Fumed silicas are available in untreated form, or with a surface treatment to render the silica more hydrophobic. Although either type can be used, preferably the fumed silica used in the present invention is untreated. The surface area of the fumed silica is preferably between about 90 to about 380 m²/g, and most preferably is between about 200 to about 380 m²/g. A particularly useful fumed silica is commercially available from Cabot Corporation under the trade name Cab-o-Sil M-5. The gellant is employed in an amount of about 0.5 to about 20% by weight of the total composition, and preferably is used in an amount of about 1% to about 10%, most preferably about 1% to about 5%. As a more specific guideline, with a higher viscosity solvent, less fumed silica is required, whereas with a lower viscosity solvent, amounts at the higher end of the range may be used.

Gelling of the fluorocarbon-containing solvent is achieved simply by combining the gellant, along with any other powder component, with the liquid solvent portion of the composition, by simple mixing by hand, or by blending in an Osterizer or equivalent blender.

It is also possible to utilize a co-gellant with the fumed silica. Although the co-gellant cannot function on its own in gelling the polyfluorinated solvent, it can contribute to the gelling function, and can aid in reducing raspiness or dryness that might be experienced when using larger quantities of fumed silica. Particularly preferred co-gellants for the present invention are dimethicone crosspolymers. A wide variety of materials of this type are available commercially, for example from Shin-Etsu. Preferred for use in anhydrous systems of the invention are vinyl dimethicone crosspolymers, in powder form. A particularly preferred material is vinyl dimethicone/methicone silsesquioxane crosspolymer. In water-containing systems of the invention, it is preferred to use fluorinated dimethicone crosspolymers. Particularly preferred for this purpose is a combination of fluorinated dimethicone crosspolymer, namely trifluoropropyl cyclopentasiloxane/trifluoropropyl cyclotetrasiloxane/trifluoropropyl dimethicone crosspolymer combined with trifluoropropyl cyclopentasiloxane/PEG-10/trifluoropropyl dimethicone crosspolymer/trifluoropropyl cyclotetrasiloxane. In contrast to the anhydrous product, these materials are preferably incorporated into the formula in the form of a gel rather than a powder. The absolute amount of co-gellant, if employed, is not crucial, and can be present in an amount of from about 0.01 to about 10%, preferably from about 0.5 to about 5%, by weight of the crosspolymer. In relation to the amount of the primary gellant, the ratio of the two will normally be approximately 1:1, if the co-gellant is used, but the co-gellant may be used in ratio as high as about 3 parts co-gellant to 1 part primary gellant with acceptable results.

An optional component of the formulation are spherical powders which can aid in enhancing the feel of the product, as well as potentially adding a “soft focus” function. Many such materials are known in the cosmetic industry for their light-scattering properties on the skin. Powders of this type may include, but are not limited to, powders comprising calcium aluminum borosilicate, polymethyl methacrylate (PMMA), polyethylene, spherical silica, methyl methacrylate crosspolymer, nylon-12, polystyrene, or ethylene/acrylic acid copolymer. Particle size of these powders range from about 5 to about 20 microns. These powders, when used, are present in an amount of from about 0.001% to about 20%, preferably about 1% to about 10%, by weight of the total composition. Not only do the powders themselves provide a smooth feel to the product on the skin when applied, but they can also, to the extent they are so adapted, be used to incorporate other desirable components for the composition, such as actives or emollients. As an example of this dual function, particularly preferred for use in the anhydrous embodiment of the invention are PMMA beads in which perfluoropolymethyl isopropyl ether (available from Cardre, Inc., South Plainfield, N.J.) is incorporated. The latter component provides an emollient effect to the skin when rubbed out, and is also compatible with the polyfluorinated solvents. In the water-containing embodiment of the invention, spherical silica particles are preferred. Preferably, the silica particles are present in a range of sizes, from about 1μ to about 100μ, preferably about 1μ to about 50μ. The silica particles may or may not be surface-treated. A particularly preferred combination of particles are silica particles having average diameters of about 3μ, 20μ, 40μ and 50μ. Commercially available combinations of this type are available under the trade name DSPCS/3H-12 and DSPCS/20N-12 from Kobo. The use of this combination of silica beads provides a smooth and silky feel to the composition, and also aids in the prevention of peeling and flaking of the product once it is rubbed out to a powder.

The present system, particularly with the use of perfluorocycloalkanes and hydrofluoroethers, is well-suited for use with soft focus powders. Such powders are characterized by being transparent, with a refractive index of about 1.5 , approximately the same as that of skin, and on the skin, they function in minimizing the appearance of lines and wrinkles by scattering and blurring the light. Similarly, most cosmetic fluids, i.e., the commonly used non-volatile oil components of liquid cosmetic products, such as esters and silicone, also have refractive indices of approximately 1.5. When soft focus powders are added into these fluids, the similarity of the refractive indices between fluid and powder results in an alteration in the way that the powders scatter light, thereby potentially diminishing their efficacy. In contrast, the perfluorocycloalkanes have a refractive index of 1.2-1.3, which means that there is enough difference between fluid and powder to permit interaction between the powder and the light, maintaining more of the benefit of the soft focus powders even with a certain amount of non-volatile fluid being present. Thus, the soft focus powders in a product of the present invention can tolerate a certain amount of nonvolatile oil in the formulation without substantially altering their effect.

Another nonessential, but sometimes desirable component, is one or more emollients, skin conditioning agents, skin protectants, or moisturizers, i.e., cosmetic materials that coat, adhere or absorb onto the skin, to enhance the smooth application and feel of the product and also to prevent a feeling of dryness. Examples of useful components of this type are medium to high molecular weight oils and esters, waxes or wax-like substances, silicone elastomer gels, perfluorpolyethers, or water. A more extensive list of ingredients of this type can be found in the International Cosmetic Ingredient Dictionary and Handbook, Eighth Edition, Volume 2, the contents of which are incorporated herein by reference. The inclusion of this type of ingredient can be useful in improving the feel and appearance of the final product; however, it is important, in the formulation of the composition, that no more than 50% non-volatile oil components be incorporated, as the low level of non-volatile oils is an important aspect of the invention. By “non-volatile oil” is meant an oil that, in contrast to the polyfluorinated solvents used, does not flash off quickly from the skin. Examples of such oils are vegetable oils, carboxylic acid esters, animal oils, glyceryl esters, non-volatile silicones, non-volatile polyfluorinated solvents, and nonvolatile hydrocarbons, such as isoparaffins, mineral oil, squalane, or petrolatum, which in many types of compositions are employed at high levels as emollients. In the present compositions, however, the composition will contain no more than about 50% non-volatile oil, preferably, no more than about 30%, more preferably no more than about 15%, most preferably no more than about 10%. In some embodiments, the composition will contain substantially no, i.e., less than 1%, non-volatile oil. Control of the amounts of these oils in the composition is important because use of higher levels of these materials may interfere with the desired rapid conversion from liquid to powder, and also may impair the light, dry, powdery feel of the product on the skin.

The composition of the invention can be used as the vehicle for any type of cosmetic or pharmaceutical composition used for topical application to the skin. As a cosmetic composition, it can be used as the base for a color cosmetic or a skin care product. In the case in which the gel is used for a color cosmetic, the base will also include one or more types of pigments. The amount of pigment used is not critical, and will depend largely on the type and intensity of color desired. Ordinarily, the pigments will be used in an amount of about 1 to about 20% by weight. The types of pigments that are employed can be any that are ordinarily used for this purpose; for example, they may be organic, including natural colorants and synthetic monomeric and polymeric colorants. Exemplary organic pigments are phthalocyanine blue and green pigment, diarylide yellow and orange pigments, and azo-type red and yellow pigments such as toluidine red, litho red, naphthol red and brown pigments. Also useful are lakes, which are pigments formed by the precipitation and absorption of organic dyes on an insoluble base, such as alumina, barium, or calcium hydrates. Particularly preferred lakes are primary FD&C or D&C lakes and blends thereof. Stains, such as bromo dyes and fluorescein dyes can also be employed.

The pigments can also be inorganic; inorganic pigments include iron oxides (yellow, red, brown or black), ferric ammonium ferrocyanide (blue), manganese violet, ultramarine blue, chrome oxide(green), talc, lecithin modified talc, zeolite, kaolin, lecithin modified kaolin, titanium dioxide(white), zinc oxide and mixtures thereof. Also useful are transparent metal oxide-coated silica beads. Metal oxides, particularly iron and titanium oxides, are preferred pigments in the composition of the invention. The pigments employed may be coated or uncoated. However, for use in a water-containing embodiment of the invention, a particularly preferred type of pigment is one which is coated with dimethicone copolyol. Such pigments are available from, for example, Cardre Inc., South Plainfield, N.J., under the name “AQ”.

The compositions of the invention in their preferred form constitute a simple anhydrous dispersion of the fumed silica particles and, if used, the pigment particles. However, the composition may in certain embodiments also contain moderate amounts of water, or can be used as the oil phase of a water and oil dispersion or emulsion. either alone, or in combination with other volatile or non-volatile oils. The preferred water-containing embodiment is a dispersion of the gel in a continuous water phase, which because of the gelling, is adequately stable even in the absence of emulsifiers. Though water-in-oil compositions may be made, the components required present a greater challenge in terms of compatibility with the polyfluorinated solvent. If the composition contains water, it will ordinarily be in an amount of from about 10% to about 60%, by weight, and preferably from about 20% to about 50%. The stability of the water-containing dispersions or emulsions can be enhanced by the incorporation of one or more self-emulsifying oils. One example of such oils is PEG-7 olivate. The self-emulsifying oil may be employed in an amount of about 3 to about 10%, keeping in mind the necessity to maintain a relatively low level of nonvolatile oils overall in the compositions.

The compositions of the invention may be used as a base for color cosmetics, for example, foundations, eyeshadows, blushes, bronzers, concealers, and the like. It may also provide a vehicle for delivery of moisturizers, emollients and/or active ingredients for a skin care or pharmaceutical product. Examples of active ingredients that can be delivered using the compositions of the invention include, but are not limited to, topically active agents that improve or eradicate age spots, keratoses and wrinkles, analgesics, anesthetics, anti-acne agents, antibacterials, antiyeast agents, antifungal agents, antiviral agents, antidandruff agents, antidermatitis agents, antipruritic agents, antiemetics, antimotion sickness agents, anti-inflammatory agents, antihyperkeratolytic agents, anti-dry skin agents, antiperspirants, antipsoriatic agents, antiseborrheic agents, antiaging agents, antiwrinkle agents, antiasthmatic agents and bronchodilators, sunscreen agents, antihistamine agents, skin lightening agents, depigmenting agents, wound-healing agents, vitamins, corticosteroids, self-tanning agents, antioxidants, free-radical scavengers, or hormones. More specific examples of useful active agents include retinoids, topical cardiovascular agents, clotrimazole, ketoconazole, miconozole, griseofulvin, hydroxyzine, diphenhydramine, pramoxine, lidocaine, procaine, mepivacaine, monobenzone, erythromycin, tetracycline, clindamycin, meclocyline, hydroquinone, minocycline, naproxen, ibuprofen, theophylline, cromolyn, albuterol, retinol, retinoic acid, 13-cis retinoic acid, hydrocortisone, hydrocortisone 21-acetate, hydrocortisone 17-valerate, hydrocortisone 17-butyrate, betamethasone valerate, betamethasone diproprionate, triamcinolone acetonide, fluocinonide, clobetasol, proprionate, benzoyl peroxide, crotamiton, propranolol, promethazine, vitamin A palmitate, vitamin E acetate, DHEA and derivatives thereof, alpha- or beta-hydroxy acids, and mixtures thereof. The amount of active agent to be used in any given formulation is readily determined in accordance with its usual dosage. In the anhydrous embodiment of the invention, tile powder form of actives can be readily accommodated.

Unlike other liquid-to-powder products, which can be more fluid and gritty, the compositions of the present invention have a luxurious creamy texture, approximating the feel of traditional creams. Also unlike other liquid-to-powder products the evaporation of the liquid base is much more thorough, leaving a drier powder on the skin than was achieved with previous products.

The invention is further illustrated by the following non-limiting examples.

### EXAMPLES

All formulas disclosed, unless otherwise stated, are made by combining the powder portion of the formula in an Osterizer blender, adding the fluid components to the blender, and blending until uniform.

I. The following formulas illustrate compositions of the invention in which polyfluorinated solvents are used alone:

| Material | Weight % |
|---|---|
| A. | |
| Titanium dioxide | 1.00 |
| Titanium dioxide/mica/silica | 4.00 |
| Silica/ethylene/methacrylate copolymer/isopropyl | 0.50 |
| titanium triisostearate (DSPCS/3H-12) | |
| Silica/ethylene/methacrylate copolymer/isopropyl | 0.50 |
| titanium triisostearate (DSPCS/20N-12) | |
| Fumed silica | 3.00 |
| propylene glycol ceteth-3 acetate | 5.00 |
| polymethyl methacrylate/ | 1.00 |
| perfluoropolymethylisopropyl ether | |
| perfluoro-1,3-dimethylcyclohexane | 85.00 |
| B. | |
| perfluoro-1,3-dimethylcyclohexane | 35.00 |
| perfluoromethyldecalin | 53.50 |
| Silica/ethylene/methacrylate copolymer/isopropyl | 1.25 |
| titanium triisostearate (DSPCS/3H-12) | |
| Silica/ethylene/methacrylate copolymer/isopropyl | 1.25 |
| titanium triisostearate (DSPCS/20N-12) | |
| Perfluoropolymethylisopropyl ether | 3.00 |
| Fumed silica | 1.00 |
| titanium dioxide | 4.15 |
| yellow iron oxide | 0.50 |
| red iron oxide | 0.30 |
| black iron oxide | 0.05 |
| C. | |
| Fumed silica | 2.75 |
| Polymethyl methacrylate/ | 3.40 |
| perfluoropolymethylisopropyl ether | |
| Mica/bismuth oxychloride/iron oxides (brown) | 3.00 |
| Mica/bismuth oxychloride/iron oxides (mauve) | 0.40 |
| Methoxynonafluorobutane* | 90.45 |

*This formula is also prepared using 2-trifluoromethyl-3-ethoxydodecafluorohexane in the same amounts.

11. The following formula illustrates a composition containing a co-gellant with the fumed silica.

| Material | Weight % |
|---|---|
| Polymethyl methacrylate/perfluoropolymethyl | 3.38 |
| isopropyl ether | |
| Mica/bismuth oxychloride/iron oxides (brown) | 3.00 |
| Mica/bismuth oxychloride/iron oxides (mauve) | 0.37 |
| Perfluoro-1,3-dimethylcyclohexane | 89.75 |
| Fumed silica | 1.50 |
| Vinyl dimethicone/methicone silsesquioxane | 2.00 |
| crosspolymer | |

III. This example illustrates a composition of the invention in which eater is present in the formula:

| Material | Weight % |
|---|---|
| Phase I | |
| Iron oxide (red)/PEG-12 dimethicone | 0.51 |
| Iron oxide (yellow)/PEG-12 dimethicone | 1.05 |
| Iron oxide (black)/PEG-12 dimethicone | 0.20 |
| Titanium dioxide)/PEG-12 dimethicone | 8.00 |
| Phase II | |
| Purified water | 38.24 |
| Phenoxyethanol | 0.80 |
| Potassium sorbate | 0.20 |
| Imidazolidinyl urea | 0.30 |
| Phase III | |
| PEG-7 olivate | 4.43 |
| Phase IV | |
| Mica/silica | 9.77 |
| Phase V | |
| Fumed silica | 1.00 |
| Phase VI | |
| Trifluoropropyl cyclopentasiloxane/trifluoropropyl | 2.00 |
| cyclotetrasiloxane/trifluoropropyl dimethicone | |
| crosspolymer | |
| Trifluoropropyl cyclopentasiloxane/PEG-10/ | 2.00 |
| trifluoropropyl dimethicone crosspolymer/ | |
| trifluoropropyl cyclotetrasiloxane | |
| Fumed silica | 1.00 |
| Phase VII | |
| Perfluoro-1,3-dimethylcyclohexane | 26.50 |
| Fumed silica | 1.00 |
| Phase VIII | |
| Silica/ethylene/methacrylate copolymer/isopropyl | 1.00 |
| titanium triisostearate (DSPCS/20N-12) | |
| Silica | 2.00 |

Phases I-IV are propeller mixed in a main beaker in a cold process. Once the materials are completely dispersed, Phases V and VI are added while continuing propeller mixing. In a separate beaker, Phase VII ingredients are mixed forming a gel. Phase VII is added to the main beaker under a Lightnin mixer, then Phase VIII is added. Once the batch is homogeneous, the batch is mixed in a Silverson mixer for 10 minutes in a cold bath.

IV. The following formula illustrates a composition comprising a combination of perfluorinated solvents and a secondary solvent.

| Material | Weight % |
|---|---|
| A. | |
| Silica/ethylene/methacrylate copolymer/isopropyl | 2.00 |
| titanium triisostearate (DSPCS/3H-12) | |
| Silica/ethylene/methacrylate copolymer/isopropyl | 2.00 |
| titanium triisostearate (DSPCS/20N-12) | |
| Phenyltrimethicone/polysilcone-11 | 2.00 |
| Fumed silica | 4.00 |
| titanium dioxide/triethoxycaprylylsilane | 4.30 |
| yellow iron oxide/triethoxycaprylylsilane | 0.40 |
| red iron oxide/triethoxycaprylylsilane | 0.26 |
| black iron oxide/triethoxycaprylylsilane | 0.04 |
| dimethicone, 20 cs | 31.00 |
| dimethicone, 5 cs | 34.00 |
| perfluoro-1,3-dimethylcyclohexane | 20.00 |
| B. | |
| Silica/ethylene/methacrylate copolymer/isopropyl | 2.00 |
| titanium triisostearate (DSPCS/3H-12) | |
| Silica/ethylene/methacrylate copolymer/isopropyl | 2.00 |
| titanium triisostearate (DSPCS/20N-12) | |
| Fumed silica | 4.00 |
| titanium dioxide/triethoxycaprylylsilane | 1.40 |
| D&C Red No. 7/methicone | 0.10 |
| Mica/titanium dioxide/methicone | 0.76 |
| red iron oxide/dimethicone | 0.45 |
| yellow iron oxide/dimethicone | 0.18 |
| black iron oxide/dimethicone | 0.11 |
| dimethicone, 5 cs | 67.00 |
| Phenyltrimethicon/polysilicone-11 | 2.00 |
| perfluoro-1,3-dimethylcyclohexane | 20.00 |

The formula as disclosed above is also made with a combination of 5 and 10 cs dimethicone, in a percent ratio of 34% and 33%, respectively, in place of the 67% of 5 cs dimethicone.

What we claim is:

1. A fluid-to-powder cosmetic or pharmaceutical composition for topical application to skin, the composition comprising at least one polyflurorinated solvent gelled with a fumed silica.

2. The composition of claim 1 in which the solvent is selected from the group consisting of perfluorocycloalkanes hydrofluoroethers, perfluoromorpholines, and perfluoroalkanes.

3. The composition of claim 1 in which the polyfluorinated solvent is a perfluorocycloalkane or hydrofluoroether.

4. The composition of claim 3 in which the polyfluorinated solvent is a perfluorocycloalkane.

5. The composition of claim 3 in which the polyfluorocycloalkane has a molecular weight between about 400 and 650, a vapor pressure between about <1 to about 50 mbar, and a boiling point ranging from about 100° C. to about 220° C.

6. The composition of claim 5 in which the perfluorocycloalkane comprises perfluorodecalin, perfluoromethyldecalin, perfluorohydrophenanthrene, or perfluoro-1,3-dimethylcyclohexane, or a combination thereof.

7. The composition of claim 6 in which the polyfluorinated solvent comprises perfluoro-1,3-dimethylcyclohexane.

8. The composition of claim 1 which comprises less than about 50% non-volatile oil.

9. The composition of claim 8 which comprises less than about 30% non-volatile oil.

10. The composition of claim 8 which comprises less than about 15% non-volatile oil.

11. The composition of claim 1 which comprises at least one secondary volatile solvent.

12. The composition of claim 11 in which the secondary solvent is selected from the group consisting of cyclic and linear silicones, straight or branched chain hydrocarbons having from 8-20 carbon atoms, and C8-20 isoparaffins.

13. The composition of claim 1 in which the gellant is an untreated fumed silica.

14. The composition of claim 13 in which the gellant has a surface area of about 200 to about 380 m²/g.

15. The composition of claim 13 in which the gellant has a surface area of about 200 m²/g.

16. The composition of claim 13 in which the gellant is present in an amount of about 0.5 to about 20% by weight of the fluid composition.

17. The composition of claim 13 in which the gellant is present in an amount of about 1 to about 5% by weight of the fluid composition.

18. The composition of claim 1 which also comprises a dimethicone crosspolymer co-gellant.

19. The composition of claim 1 which is an anhydrous composition.

20. The composition of claim 1 which is a water-containing composition.

21. The composition of claim 19 which contains a vinyl dimethicone crosspolymer co-gellant.

22. The composition of claim 20 which contains a fluorinated dimethicone crosspolymer.

23. A fluid-to-powder cosmetic or pharmaceutical composition for topical application to skin, the composition comprising at least one polyfluorocycloalkane solvent gelled with a fumed silica.

24. The composition of claim 23 which comprises less than about 50% by weight of a non-volatile oil.

25. The composition of claim 23 which comprises less than about 30% by weight of a non-volatile oil.

26. The composition of claim 24 in which the polyfluorocycloalkane is present in an amount of about 40 to about 98% by weight of the composition and the fumed silica is present in an amount of about 1 to about 5%.

27. The composition of claim 23 which also comprises at least one pigment.

28. The composition of claim 23 which is a water-containing composition.

29. The composition of claim 27 which is a water-containing composition, and the pigment is coated with dimethicone copolyol.

30. The composition of claim 23 which contains a dimethicone crosspolymer co-gellant.

31. The composition of claim 30 which is anhydrous, and contains a vinyl dimethicone crosspolymer co-gellant.

32. The composition of claim 30 which is a water-containing composition, and contains a fluorinated dimethicone crosspolymer.

33. The composition of claim 23 which also contains a soft-focus powder.

34. The composition of claim 33 in which the powder comprises polymethylmethacrylate.
